(19) United States
(12) Patent Application Publication
(10) Pub. No.: US 2023/0165470 A1
(43) Pub. Date: Jun. 1, 2023

(54) HEALTH MONITOR FOR A NON-HUMAN ANIMAL

(71) Applicant: PETMEDICS LLC, New York, NY (US)

(72) Inventors: Jack N. MIZRAHI, New York, NY (US); Ezra S. ASHKENAZI, Rahway, NJ (US)

(21) Appl. No.: 18/072,317

(22) Filed: Nov. 30, 2022

Related U.S. Application Data
(60) Provisional application No. 63/284,971, filed on Dec. 1, 2021.

(51) Int. Cl. A61B 5/01; A61B 5/00

(52) U.S. Cl. CPC A61B 5/01; A61B 5/746; A61B 5/742; A61B 2503/40

(57) ABSTRACT

In one embodiment, a health monitor for a warm-blooded non-human animal is disclosed. The health sensor is configured to capture health information of the non-human animal. The health monitor includes a display including a light source. The health monitor includes and an evaluating module configured to compare the health information to a first threshold based on a hypothermic condition of the non-human animal, generate a comparison that determines if the health information is less than or equal to the first threshold, and based on the comparison, generate a first alert.

### CROSS-REFERENCE TO RELATED APPLICATIONS

This application claims the benefit of priority pursuant to 35 U.S.C. § 119(e) of U.S. Provisional Patent Application No. 63/284,971, filed Dec. 1, 2021, entitled “HEALTH MONITOR FOR A NON-HUMAN ANIMAL, which his hereby incorporated by reference herein in its entirety.

### BACKGROUND

Health monitors, such as non-contact thermometers are well known for use in industrial and human healthcare applications. Health monitors may capture and record data related to a variety of health conditions. For example, thermometers are used to detect febrile conditions in patients. In some instances, thermometers adapted to detect febrile conditions in humans are used in non-human animals such as pets or livestock. Such approaches have a number of drawbacks. For example, the range of healthy body temperatures may vary based on the species of animal whose temperature is being measured. A thermometer adapted for human use may alarm at temperatures that are abnormal for humans but are normal for other animals. Thus, if a user suspects a non-human animal is ill and measures the non-human animal's temperature with a thermometer adapted for human use, the thermometer may cause false alarms, or may fail to alarm when it should, because the thermometer is not adapted for use with the species of non-human animal being monitored. In another example, non-contact thermometers adapted for human use may perform calculations that correlate a measured skin temperature for a human to an estimated body temperature. Such calculations may be inapt for non-human animals. A dedicated health monitor for non-human animals is therefore needed.

### BRIEF SUMMARY

In one embodiment, a health monitor for a warm-blooded non-human animal is disclosed. The health sensor is configured to capture health information of the non-human animal. The health monitor includes a display including a light source. The health monitor includes and an evaluating module configured to compare the health information to a first threshold based on a hypothermic condition of the non-human animal, generate a comparison that determines if the health information is less than or equal to the first threshold, and based on the comparison, generate a first alert.

Optionally, in some embodiments, the health sensor includes a non-contact thermometer.

Optionally, in some embodiments, the display is configured to display the first alert.

Optionally, in some embodiments, the evaluating module generates the first alert when the health information is less than or equal to the first threshold.

Optionally, in some embodiments, the evaluating module is configured to compare the health information to a second threshold, generate a second comparison that determines if the health information is greater than the first threshold and less than or equal to the second threshold, and generate a safe message based on the second comparison.

Optionally, in some embodiments, the display is selectively configurable to display historical health information.

Optionally, in some embodiments, the first threshold, the second threshold, and the third threshold are selected based on a type of non-human animal.

Optionally, in some embodiments, the light source is configured to illuminate with a first color based on the comparison.

Optionally, in some embodiments, the light source is configured to illuminate with a second color based on the second comparison.

Optionally, in some embodiments, the evaluating module is configured to compare the health information to a third threshold, generate a third comparison that determines if the health information is greater than the second threshold and less than or equal to the third threshold, and generate a monitoring message based on the third comparison.

Optionally, in some embodiments, the light source is configured to illuminate with a third color based on the third comparison.

Optionally, in some embodiments, the evaluating module is configured to generate a fourth comparison that determines if the health information is greater than the third threshold, and generate a second alert message based on the fourth comparison.

Optionally, in some embodiments, the light source is configured to illuminate with the first color based on the fourth comparison.

In one embodiment, a method of monitoring the temperature of a pet is disclosed. The method includes receiving, at an evaluating module via a temperature sensor, temperature information of the pet; determining, via the evaluating module, whether the temperature information is greater than a first febrile temperature threshold specific to a pet type, wherein the pet type is not human; and generating, via the evaluating module, an alert for the type of pet based on the first febrile temperature specific to the pet type.

Optionally, in some embodiments, the method includes displaying via a display the first alert.

Optionally, in some embodiments, the method includes illuminating a light source with a first color based on the alert.

Optionally, in some embodiments, the method includes determining, via the evaluating module, whether the temperature information is greater than a second febrile temperature threshold specific to the pet type and less than or equal to the first febrile temperature threshold; and generating, via the evaluating module, a monitoring message for the type of pet based on the first febrile temperature and the second febrile temperature specific to the type of pet.

Optionally, in some embodiments, the method includes illuminating a light source with a second color based on the monitoring message.

Optionally, in some embodiments, the method includes determining, via the evaluating module, whether the temperature information is greater than a first afebrile temperature threshold specific to the pet type and less than or equal to the second febrile temperature threshold; and generating, via the evaluating module, a safe message for the type of pet based on the first afebrile temperature and the second febrile temperature specific to the type of pet.

Optionally, in some embodiments, the method includes illuminating a light source with a third color based on the safe message.

Optionally, in some embodiments, the method includes determining, via the evaluating module, whether the temperature information is less than or equal to the first afebrile temperature threshold; generating, via the evaluating module, a second alert message for the type of pet based on the first afebrile temperature specific to the type of pet.

### BRIEF DESCRIPTION OF THE DRAWINGS

The drawings are not necessarily to scale. In certain instances, details unnecessary for understanding the disclosure or rendering other details difficult to perceive may have been omitted. In the appended drawings, similar components and/or features may have the same reference label. Further, various components of the same type may be distinguished by following the reference label by a letter or number that distinguishes among the similar components. The claimed subject matter is not necessarily limited to the particular examples or arrangements illustrated herein.

FIG. 1 is a perspective view of an embodiment of a health monitor adapted for use with a non-human animal.

FIG. 2 is a rear elevation view of the health monitor of FIG. 1.

FIG. 3A is a front elevation view of the health monitor of FIG. 1 in a first configuration.

FIG. 3B is a front elevation view of the health monitor of FIG. 1 in a second configuration.

FIG. 3C is a front elevation view of the health monitor of FIG. 1 in a third configuration.

FIG. 4 is a perspective view of a user using the health monitor of FIG. 1.

FIG. 5 is a flowchart of a method of monitoring health information of a patient with the health monitor of FIG. 1.

FIG. 6 is a partial detail view of historical information displayed on a display of the health monitor of FIG. 1.

FIG. 7 is a simplified block diagram of components of the health monitor of FIG. 1.

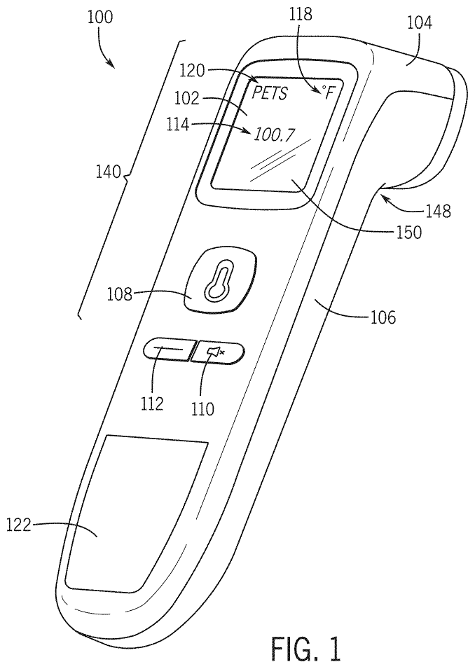

### DETAILED DESCRIPTION

The present disclosure relates to an animal health monitor 100 adapted for use with a non-human animal. The animal health monitor 100 is adapted to measure health information 114 of a non-human animal and to interpret the measured health information 114 based on the species of non-human animal being monitored. In many embodiments, the animal health monitor 100 includes a health sensor 125 that detects the health information 114. In some embodiments, the health monitor 100 or thermometer may generate output information specific to the non-human animal being assessed. In many embodiments, the monitor 100 may output a “no fever” or other indicator of healthy status for temperature ranges that would be considered to be febrile for humans. The specific tailoring of the outputs allows users to accurately assess pets, such as dogs and cats, for health issues, without resulting in false alarms. Conventionally, pet owners and/or pet care givers, such as veterinarians, must use thermometers and other health devices designed for humans to detect health information from pets. This may cause inadvertent panic, expense in veterinary bills, and the like. The animal health monitor 100 of the present disclosure avoids possible false alarms, reducing panic and unnecessary treatment, by being calibrated to generate alerts based on temperatures that are actually indicative of illness in a pet, such as a dog or cat. Health monitors adapted for human use cannot be used for non-human animals, because they will provide alerts that are for the wrong ranges of body temperatures. Human health monitors that don't provide alerts will result in user error as the humans may interpret health information readings, such as temperature, against known thresholds for humans (e.g., 98.6° F. as a normal human body temperature) and will not know that a pet is sick (e.g., a temperature of 98.6° F. may be abnormally low for a dog). Alternately, a user reading a normal non-human animal temperature of about 100° F. may be falsely alarmed at what the user thinks is an abnormal temperature, resulting in panic, un-necessary veterinarian visits, costs, and the like.

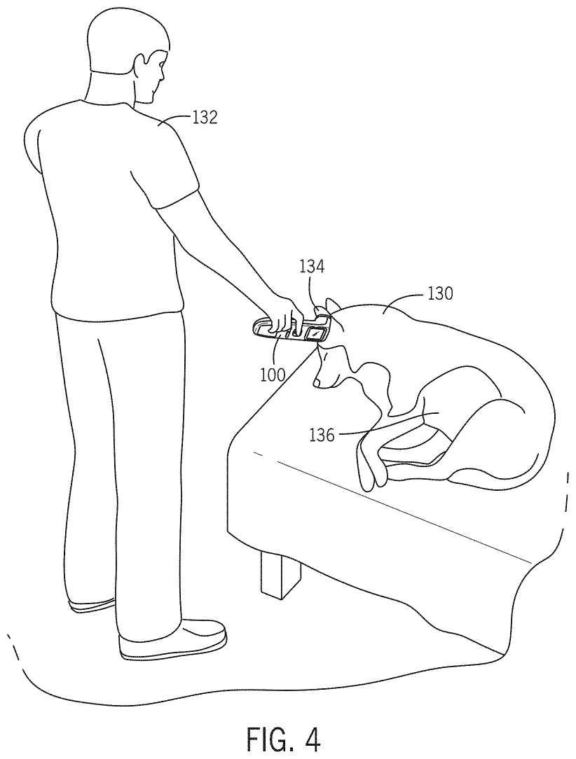

In many embodiments, the animal health monitor 100 includes a housing adapted to be comfortably held in a hand of a human user 132 for use for monitoring a non-human animal or animal patient 130 (shown for example as a dog in FIG. 4). In many examples of using the animal health monitor 100, the animal patient 100 will be warm-blooded, such as a mammal or bird. In many embodiments, the animal health monitor 100 is a pet thermometer 100 adapted for use with household pets such as dogs, cats, Guinea pigs, birds, or the like. The housing 148 may be formed from an upper housing 104 and a lower housing 106. The upper housing 104 and lower housing 106 may be couplable to one another with a suitable fastener such as one or more screws, rivets, an adhesive, or the like. In some embodiments, the upper housing 104 and lower housing 106 may snap or click together to form a housing 148 that is not readily separable.

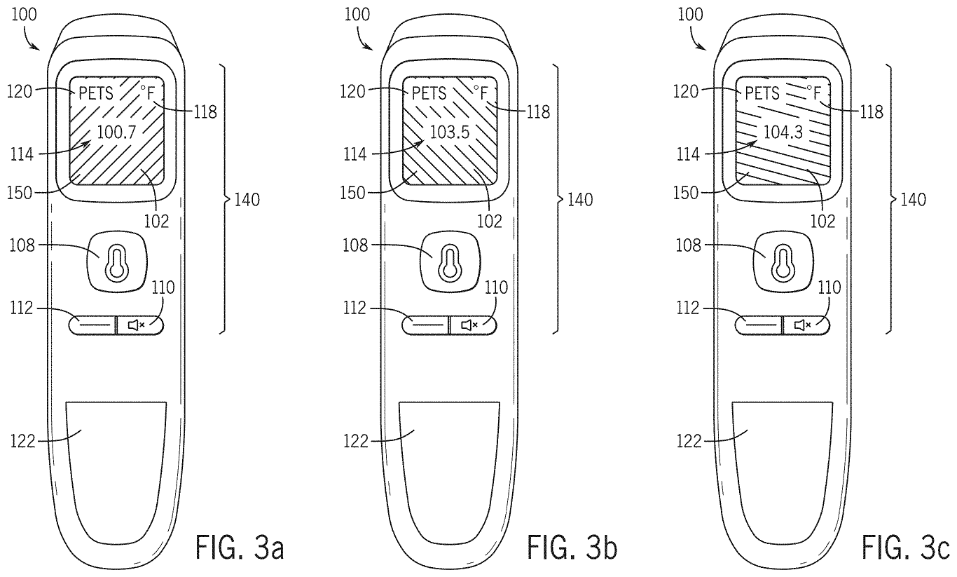

The animal health monitor 100 includes an input/output interface 140 that receives inputs from the user 132 and displays or otherwise notifies the user of health information 114 detected by the animal health monitor 100. The input/output interface 140 may include a display 102 that outputs visual information, such as text and/or hues, to the user. The display 102, may be, for example, a liquid crystal display (“LCD”), light emitting diode (“LED”) display, or the like that displays visual information to the user 132. The display 102 may include a light source 150 such as a multi-colored backlight (as discussed below and shown for example, in FIG. 3A-FIG. 3C). In some embodiments, the light source 150 may embodiment separate from the display. The light source 150 may be configured to communicate alerts to the user 132.

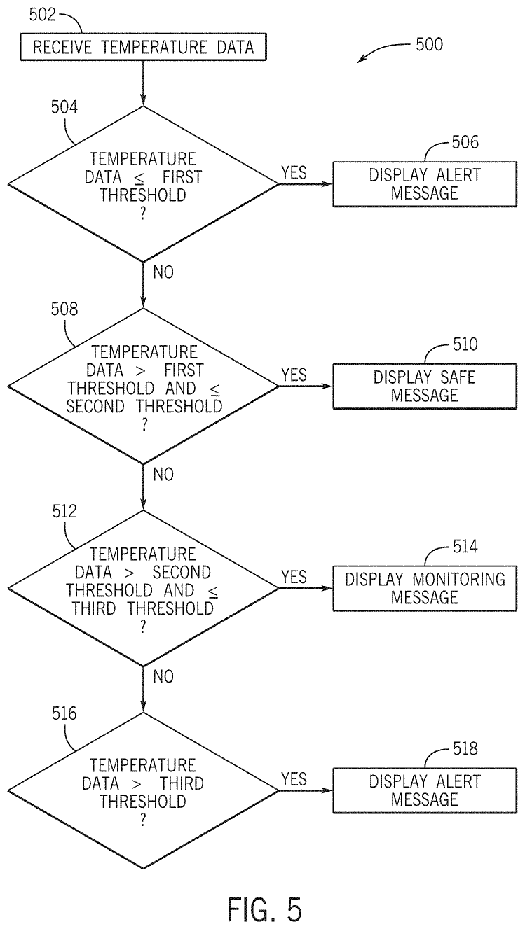

The display 102 may include areas for displaying different types of information. For example, the display 102 may include a region that displays health information 114 such as a body or skin temperature reading of an animal patient 130. The display 102 may include a region that displays patient type 120 information, such as “Pet”. The patient type 120 may also include a type of animal patient 130 such as “dog”, “cat”, “bovine”, “equine”, “rodent”, “reptile”, “porcine”, “goat”, “sheep”, etc. The patient type 120 may be selectable by the user, via one or more inputs in the input/output interface 140. In some embodiments, when a patient type 120 is selected, the evaluating module 138 may receive an input from any of the portions of the input/output interface and select appropriate thresholds (such as the thresholds discussed with respect to FIG. 5 and the method 500) for normal and abnormal ranges of the health information 114, such as by reading from a list of pre-determined thresholds stored in a memory component 142.

The input/output interface 140 may include an actuator 108 that activates the animal health monitor 100 to cause it to detect health information 114 via the health sensor 125. In some embodiments, the actuator 108 may be a physical button that detects a press from a user 132. The actuator 108 may be substantially any type of sensor that can detect a user input, such as a soft (or virtual) button on a touch screen, a capacitive sensor, a resistive sensor, a switch, a proximity detector, or other suitable device that can detect a user's command to activate the animal health monitor 100. In some embodiments, the actuator 108 may have different functions depending on how long or how many times it is pressed. For example, a press-and-hold of the actuator 108 may cause the animal health monitor 100 to receive health information 114 via the health sensor 125. The display 102 may include a region that displays units information 118, such as a unit of measurement of the health information 114 (e.g., ° F. or ° C.). In some embodiments, a short press of the actuator 108 may cause the animal health monitor 100 to display or output health information 114 in different units (e.g., ° F. or ° C.). In some embodiments, the actuator 108 may be adapted to enable a user to select a patient type 120 such as “dog”, “cat”, “bovine”, “equine”, “rodent”, “reptile”, etc. The animal health monitor 100 may automatically adjust calculations and/or alert levels based on a patient type 120 selected.

While in many embodiments, the health sensor 125 is a non-contact temperature sensor, other health sensors 125 are envisioned for use with the animal health monitor 100 for monitoring the health of an animal patient 130. For example, a health sensor may be a pulse oximeter, blood pressure monitor, blood glucose detector, electrocardiogram, weight scale, a contact thermometer (e.g., an oral, rectal, or arterial thermometer), or the like.

The input/output interface 140 may include a notification actuator 110 that configures an output, which may be a sound or visual output, of the animal health monitor 100. For example, the animal health monitor 100 may include a sound output such as a speaker, piezoelectric element, buzzer, beeper, or the like that provides an alert to the users 132. For example a sound output may indicate when a successful reading has been taken, when an abnormal health information 114 such as body temperature has been detected, when health information 114 is normal, etc. The notification actuator 110 may also silence such sounds outputs or change the volume thereof. In another, example, the notification actuator 110 may change the brightness, hue, or other aspect the display 102 and/or the light source 150. In some examples, an alert may include a multi-mode alert that causes both the light source 150 to illuminate and causes a sound output to be emitted.

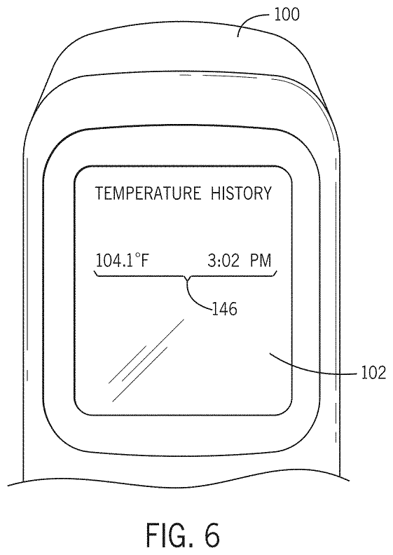

The animal health monitor 100 may include a memory component 142 that can, among other things, store historical health information 146. The input/output interface 140 may include a memory actuator 112 that causes the animal health monitor 100 to selectively display the historical health information 146. See FIG. 6 and related discussion below.

The housing 148 may include an interpretive display 122 that includes information related to normal health information 114 for an animal patient 130. For example, the interpretive display 122 may include body temperature ranges that are normal or abnormal for an animal patient 130. The interpretive display 122 may include instructions for actions to take when abnormal health information 114 is detected. For example, the interpretive display 122 may include instructions to call an animal healthcare professional (e.g., a veterinarian) if a temperature is out of a normal range.

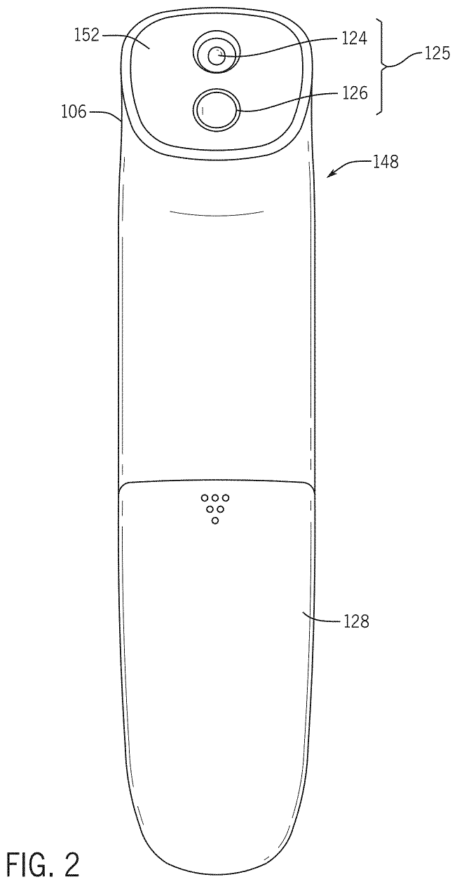

With reference to FIG. 2, a rear elevation view of the animal health monitor 100 is shown. A sensor panel 152 may be coupled to the housing 148. For example, the sensor panel 152 may be received between the upper housing 104 and the lower housing 106. The sensor panel 152 supports the health sensor 125. The sensor panel 152 may include one or more apertures formed therein may receive a respective emitter 124 and a detector 126 of the health sensor 125. The emitter 124 may emit light, such as infrared light. In use, the animal health monitor 100 may be oriented to direct the light toward an animal patient 130. The light emitted from the emitter 124 may reflect off of the skin of the animal patient 130. The reflected light may be received by the detector 126. The reflected light may include information indicative of the health information 114 of the animal patient 130, such as a skin temperature. The detector 126 may include a lens to gather and/or focus emitted light. In some embodiments, the emitter 124 is optional and the detector may detect radiation emitted (e.g., infrared light) emitted by the animal patient 130. Also shown for example in FIG. 2 is a removable cover 128 that can be selectively removed from the housing 148 to allow a user 132 to access a power source 144, such as batteries, to install, remove, or replace the same. In another embodiment, the health sensor 125 may be a contact thermometer such as a rectal, oral, or arterial thermometer that physically contacts the animal patient 130 to capture health information 114, such as a body temperature.

With reference to FIG. 3A-FIG. 3C, the animal health monitor 100 is shown in configurations that alert the user 132 to conditions of the animal patient 130 based on the health information 114, such as by the use of the light source 150. The configurations of the animal health monitor 100 shown in FIG. 3A-FIG. 3C may correspond to one or more outputs from operations of the method 500 as discussed with respect to FIG. 5.

FIG. 3A illustrates the animal health monitor 100 where the light source 150 is configured to illuminate with a first color (indicated by the hatch pattern on the display 102). The first color may indicate that the health information 114 detected by the health sensor 125 is within a normal range. Display of the first color may correspond to a safe message of operation 510, such as when the animal patient 130 is afebrile.

FIG. 3B illustrates the animal health monitor 100 where the light source 150 is configured to illuminate with a second color (indicated by the hatch pattern on the display 102). The second color may indicate that the health information 114 detected by the health sensor 125 is within a range that is not normal and should be monitored. Display of the second color may correspond to a monitoring message of operation 514.

FIG. 3C illustrates the animal health monitor 100 where the light source 150 is configured to illuminate with a third color (indicated by the hatch pattern on the display 102). The third color may indicate that the health information 114 detected by the health sensor 125 is within an alert range indicating that the animal patient 130 needs urgent medical attention, such as when the animal patient 130 is hypothermic. Display of the third color may correspond to a monitoring message of operation 506 and/or operation 518.

In some embodiments, the first, second, or third colors may be green, yellow, and red respectively. However, without limitation, any of the first, second, or third colors may be any spectrum of light perceptible to a user 132.

With reference to FIG. 4, an example of a user 132 using the animal health monitor 100 on an animal patient 130 is shown. In the example shown, the animal patient 130 is a dog. However in other examples, the animal patient 130 may be any non-human animal. The user 132 may orient the health sensor 125 toward the animal patient 130. In many examples, the user 132 may orient the health sensor 125 toward a portion of the body of the animal patient 130 that is relatively devoid of fur or hair, for example an ear 134 and/or the abdomen 136. The user 132 may hold the health sensor 125 close to the ear 134 (such as an inner ear) and/or abdomen 136 while activating the actuator 108. When activated, the actuator 108 may cause the animal health monitor 100 to read health information 114 from the health sensor 125. The animal health monitor 100 may, via an evaluating module 138, execute the method 500 discussed herein and display the health information 114.

With reference to FIG. 5, a method 500 for monitoring the health of an animal health monitor 100 is disclosed. The method 500 may begin in operation 502 and the animal health monitor 100 receives health information 114 such as temperature data, for example as shown and described with respect to FIG. 4. The operations of the method 500 may be executed in an order other than as shown, and two or more operations may be executed in parallel with one another.

The method 500 may proceed to operation 504 and the evaluating module 138 compares the health information 114 to a first threshold. If the health information 114 is less than or equal to the first threshold, the method 500 may proceed to operation 506 and the display 102 displays the health information 114 with an alert message. Such conditions may exist when the animal patient 130 is hypothermic. The user 132 may be directed to see medical care for the animal patient 130. For example, the light source 150 may illuminate with the third color. See, e.g., FIG. 3C. Optionally, the animal health monitor 100 may emit an alert sound and/or a visual alert. Any of the operations (e.g., operations 506, 510, 514, and/or 518) where the animal health monitor 100 generates an alert may be a visual alert, an auditory alert, and/or a combination thereof. If the health information 114 is not less than or equal than the first threshold, the method 500 may proceed to operation 508. In some embodiments, the first threshold is approximately 98.9° F. (37.2° C.), such as when the patient type is a dog or cat. In some embodiments, the first threshold is approximately 102.5° F. such as when the patient type is a pig. In some embodiments, the first threshold is approximately 99-101.5° F. such as when the patient type is an equine such as a horse, pony, mule, hinny, or donkey. In some embodiments, the first threshold is approximately -101.5-102.5° F. such as when the patient type is a bovine. In some embodiments, the first threshold is approximately 101.5-103.5° F. such as when the patient type is a sheep or goat. As discussed above, the patient type may be selectable via the user interface 140 such that the operations of the method 500 use thresholds, and generate messages or alerts, appropriate for the selected patient type. One or more sets of thresholds may be stored in the memory of the health monitor and retrieved by the evaluating module for use in executing the method 500. In some embodiments, one or more of the second, third or subsequent thresholds may be automatically adjusted based on a selected first threshold. In some embodiments, the patient type may include other data about the patient such as a patient age, weight, gender, etc. in addition to a species. For example, a patient type may be a foal (i.e., a young horse). The first threshold for a foal may be in the range of about 99.5-102.1° F.

In operation 508, the evaluating module 138 compares the health information 114 to the first threshold and a second threshold. If the health information 114 is greater than the first threshold and less than or equal to the second threshold, the method 500 may proceed to operation 510 and the display 102 displays the health information 114 with a safe message, such as when the animal patient 130 is afebrile. For example, the light source 150 may illuminate with the first color. See, e.g., FIG. 3A. Optionally, the animal health monitor 100 may emit a sound indicating a normal condition of the animal patient 130. In some embodiments, the second threshold is approximately 102.5° F. (39.2° C.). If the health information 114 is not less than or equal than the second threshold, the method 500 may proceed to operation 512.

In operation 512, the evaluating module 138 compares the health information 114 to the second threshold and a third threshold. If the health information 114 is greater than the second threshold and less than or equal to the third threshold, the method 500 may proceed to operation 514 and the display 102 displays the health information 114 with a monitoring message, such as when the animal patient 130 is slightly febrile. For example, the light source 150 may illuminate with the second color. See, e.g., FIG. 3B. A monitoring message may indicate that the health information 114 is abnormal but not yet to a level requiring medical intervention. A user 132 may be advised by the animal health monitor 100 to monitor the animal patient 130. Optionally, the animal health monitor 100 may emit a sound indicating that the animal patient 130 should be monitored. In some embodiments, the third threshold is approximately 104° F. (40° C.). If the health information 114 is not less than or equal than the third threshold, the method 500 may proceed to operation 516.

In operation 516, the evaluating module 138 compares the health information 114 to the third threshold. If the health information 114 is greater than the third threshold, the method 500 may proceed to operation 518 and the display 102 displays the health information 114 with an alert message, such as when the animal patient 130 is febrile to an extent that medical attention is needed. For example, the light source 150 may illuminate with the third color. See, e.g., FIG. 3C. The user 132 may be directed to see medical care for the animal patient 130. Optionally, the animal health monitor 100 may emit an alert sound.

Any of the thresholds may be stored in the memory component 142. Any of the thresholds may be varied based on the patient type 120. For example different thresholds may be used for different species of non-human animals. In some embodiments, the animal health monitor 100 may include calculations that correlate a measured skin temperature with an estimated body temperature based on the patient type 120, such as a species. For example, the evaluating module 138 may receive health information 114 such as skin temperature information and may correlate the same to an estimated internal body temperature based on a species of the animal patient 130. In some examples, the memory component 142 may store one set of thresholds (e.g., first, second, and third thresholds) that correspond to a first patient type 120 (e.g., dog, etc.) and a second or subsequent set of thresholds that correspond to a second or subsequent patient type 120 (e.g., cat, etc.). Thus, the animal health monitor 100 may have significant advantages over the use of a thermometer adapted for human use with an non-human animal.

As shown in FIG. 6, the display 102 may be configurable to display historical health information 146 including one or more health information 114 readings of the animal patient 130. For example, the memory component 142 may record health information 114 readings along with a time stamp of the reading. In some embodiments, the display 102 shows a single record of historical health information 146. In some embodiments, the display 102 shows two or more records of historical health information 146. When the user 132 activates the memory actuator 112, the evaluating module 138 may cause the display 102 to display the historical health information 146. An advantage of this historical health information 146 may be the ability to detect trends (e.g., worsening or improvement) of the condition of the animal patient 130.

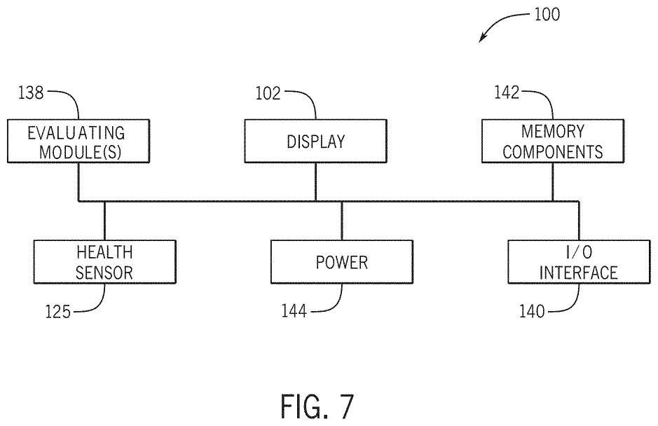

FIG. 7 illustrates a simplified block diagram for the various devices of the animal health monitor 100. As shown, the animal health monitor 100 may include one or more evaluating modules 138, a display 102, one or more memory components 142, a health sensor 125, a power source 144, and an input/output interface 140, where the various components may be in direct or indirect communication with one another, such as via one or more system buses, contract traces, wiring, or via wireless mechanisms.

The one or more evaluating modules 138 may be substantially any electronic device capable of processing, receiving, and/or transmitting instructions. For example, the evaluating modules 138 may be a microprocessor, microcomputer, graphics processing unit, an analog electrical circuit, or the like. It also should be noted that the evaluating modules 138 may include one or more evaluating modules or modules that may or may not be in communication with one another. For example, a first evaluating module may control a first set of components of the computing device and a second evaluating module may control a second set of components of the computing device where the first and second evaluating modules may or may not be in communication with each other. Relatedly, the evaluating modules may be configured to execute one or more instructions in parallel locally, and/or across a network, such as through cloud computing resources.

The display 102 provides an input/output mechanism for animal health monitor 100, such as to display visual information (e.g., images, graphical user interfaces, videos, notifications, and the like) to a user, and in certain instances may also act to receive user input (e.g., via a touch screen or the like). The display may be an LCD screen, plasma screen, LED screen, an organic LED screen, or the like. The type and number of displays may vary.

The memory component 142 stores electronic data that may be utilized by the animal health monitor 100, such as audio files, video files, document files, programming instructions, health information 114, historical health information 146, and the like. The memory components 142 may be, for example, non-volatile storage, a magnetic storage medium, optical storage medium, magneto-optical storage medium, read only memory, random access memory, erasable programmable memory, flash memory, or a combination of one or more types of memory components.

The animal health monitor 100 may also include a power source 144. The power source 144 provides power to various components of the animal health monitor 100. The power source 144 may include one or more rechargeable, disposable, or hardwire sources, e.g., batteries, power cord, AC/DC inverter, DC/DC converter, or the like. In some embodiments, the power source 144 may include a connector (such as a universal serial bus) that provides power to the computer or batteries within the computer and also transmits data to and from the device to other devices.

The input/output interface 140 allows the animal health monitor 100 to receive input from a user 132 and provide output to a user 132. For example, the input/output interface 140 may include a capacitive touch screen, keyboard, mouse, stylus, soft buttons, a touch screen, physical buttons, switches, or the like. The type of devices that interact via the input/output interface 140 may be varied as desired.

The description of certain embodiments included herein is merely exemplary in nature and is in no way intended to limit the scope of the disclosure or its applications or uses. In the included detailed description of embodiments of the present systems and methods, reference is made to the accompanying drawings which form a part hereof, and which are shown by way of illustration specific to embodiments in which the described systems and methods may be practiced. These embodiments are described in sufficient detail to enable those skilled in the art to practice presently disclosed systems and methods, and it is to be understood that other embodiments may be utilized, and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The included detailed description is therefore not to be taken in a limiting sense, and the scope of the disclosure is defined only by the appended claims.

From the foregoing it will be appreciated that, although specific embodiments of the invention have been described herein for purposes of illustration, various modifications may be made without deviating from the spirit and scope of the invention.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

What is claimed is:

1. A health monitor for a warm-blooded non-human animal comprising:
a health sensor configured to capture health information of the non-human animal;
a display including a light source; and
an evaluating module configured to:
compare the health information to a first threshold based on a hypothermic condition of the non-human animal,
generate a comparison that determines if the health information is less than or equal to the first threshold, and
based on the comparison, generate a first alert.

2. The health monitor of claim 1, wherein the health sensor comprises a non-contact thermometer.

3. The health monitor of claim 1, wherein the display is configured to display the first alert.

4. The health monitor of claim 3, wherein the light source is configured to illuminate with a first color based on the comparison.

5. The health monitor of claim 4, wherein the evaluating module is configured to:
compare the health information to a second threshold based on an afebrile condition of the non-human animal;
generate a second comparison that determines if the health information is greater than the first threshold and less than or equal to the second threshold; and
generate a safe message based on the second comparison.

6. The health monitor of claim 5, wherein the light source is configured to illuminate with a second color based on the second comparison.

7. The health monitor of claim 5, wherein the evaluating module is configured to:
compare the health information to a third threshold based on a first febrile condition of the non-human animal;
generate a third comparison that determines if the health information is greater than the second threshold and less than or equal to the third threshold; and
generate a monitoring message based on the third comparison.

8. The health monitor of claim 7, wherein the light source is configured to illuminate with a third color based on the third comparison.

9. The health monitor of claim 7, wherein the evaluating module is configured to:
generate a fourth comparison based on a second febrile condition of the non-human animal that determines if the health information is greater than the third threshold; and
generate a second alert message based on the fourth comparison.

10. The health monitor of claim 9, wherein the light source is configured to illuminate with the first color based on the fourth comparison.

11. The health monitor of claim 1, wherein the display is selectively configurable to display historical health information.

12. The health monitor of claim 7, wherein the first threshold, the second threshold, and the third threshold are selected based on a type of non-human animal.

13. The health monitor of claim 1, wherein the first threshold is about 98.9° F.

14. The health monitor of claim 5, wherein the second threshold is about 102.5° F.

15. The health monitor of claim 7, wherein the third threshold is about 104° F.

16. A method of monitoring the temperature of a pet comprising:
receiving, at an evaluating module via a temperature sensor, temperature information of the pet;
determining, via the evaluating module, whether the temperature information is greater than a first febrile temperature threshold specific to a pet type, wherein the pet type is not human; and
generating, via the evaluating module, an alert for the type of pet based on the first febrile temperature specific to the pet type.

17. The method of claim 16, wherein the health sensor comprises a non-contact thermometer.

18. The method of claim 16, further comprising displaying via a display the first alert.

19. The method of claim 18, further comprising illuminating a light source with a first color based on the alert.

20. The method of claim 18, further comprising:
determining, via the evaluating module, whether the temperature information is greater than a second febrile temperature threshold specific to the pet type and less than or equal to the first febrile temperature threshold; and
generating, via the evaluating module, a monitoring message for the type of pet based on the first febrile temperature and the second febrile temperature specific to the type of pet.

21. The method of claim 20, further comprising illuminating a light source with a second color based on the monitoring message.

22. The method of claim 20, further comprising:
determining, via the evaluating module, whether the temperature information is greater than a first afebrile temperature threshold specific to the pet type and less than or equal to the second febrile temperature threshold; and
generating, via the evaluating module, a safe message for the type of pet based on the first afebrile temperature and the second febrile temperature specific to the type of pet.

23. The method of claim 22, The method of claim 15, further comprising illuminating a light source with a third color based on the safe message.

24. The method of claim 22, further comprising:
determining, via the evaluating module, whether the temperature information is less than or equal to the first afebrile temperature threshold; and
generating, via the evaluating module, a second alert message for the type of pet based on the first afebrile temperature specific to the type of pet.

25. The method of claim 19, further comprising illuminating a light source with the first color based on the second alert.
